(19) United States
(12) Patent Application Publication
(10) Pub. No.: US 2011/0071429 A1
(43) Pub. Date: Mar. 24, 2011

(54) EUS-FNA Stylet Withdrawal into Handle

(76) Inventor: Michal Weisman, Fair Lawn, NJ (US)

(21) Appl. No.: 12/889,924

(22) Filed: Sep. 24, 2010

Related U.S. Application Data
(60) Provisional application No. 61/245,364, filed on Sep. 24, 2009.

(51) Int. Cl. A61B 10/02; A61M 1/00

(52) U.S. Cl. 600/566; 600/567

(57) ABSTRACT

A medical device comprises a needle configured for insertion into a body and defining a needle lumen extending therethrough from a proximal end opening to a distal end opening and a handle connected to a proximal end of the needle and including a stylet receiving lumen therein, the stylet receiving lumen being open to a proximal end of the needle lumen. The medical device also comprises a stylet movable between a retracted configuration in which a distal end of the stylet is received within the stylet receiving chamber and an extended configuration in which the distal end of the stylet is received within the distal opening of the needle lumen sealing the distal opening, wherein, when withdrawn proximally into the stylet receiving lumen, the stylet is coiled within the chamber.

### PRIORITY CLAIM

The present application claims the priority to the U.S. Provisional Application Ser. No. 61/245,364, entitled “EUS-FNA Stylet Withdrawal into Handle” filed on Sep. 24, 2009. The specification of the above-identified application is incorporated herewith by reference

### BACKGROUND

Biopsies may be performed via Endoscopic Ultrasound Fine Needle Aspiration (“EUS-FNA”) to obtain cells or small samples of tissue from, for example, the breast or liver for cytology studies, endoscopy or oncology. With current EUS-FNA devices, a stylet is inserted through the lumen of a needle to the distal end thereof to prevent tissue from entering the needle as the needle passes through tissue along an insertion path before a target site is reached. When the needle has reached the target site, the stylet is withdrawn proximally from the device to open the lumen. The needle is then inserted into the target tissue and negative pressure may be applied therethrough to aspirate sample tissue from the distal end of the needle further into the lumen. After the sample has been obtained and the needle has been removed from the body, the stylet is passed distally through the lumen to push the sample tissue out of the distal end of the needle (e.g., onto a slide or into another collection area). In many instances, EUS-FNA devices yield samples that are too small, which are contaminated during the biopsy procedure or which are otherwise flawed to the extent that thorough analysis and diagnosis is not possible. In these cases, the tissue must be resampled increasing the time and expense associated with the EUS-FNA procedure. Furthermore, present EUS-FNA devices require removal of the stylet in order to receive a biopsy sample. This withdrawal can cause kinking of the stylet and may result in the stylet becoming unsterile, preventing reinsertion thereof into the EUS-FNA device. In such cases, a new EUS-FNA device must be employed to acquire further biopsy samples.

### SUMMARY OF THE INVENTION

The present invention is directed to a medical device comprises a needle configured for insertion into a body and defining a needle lumen extending therethrough from a proximal end opening to a distal end opening and a handle connected to a proximal end of the needle and including a stylet receiving lumen therein, the stylet receiving lumen being open to a proximal end of the needle lumen. The medical device also comprises a stylet movable between a retracted configuration in which a distal end of the stylet is received within the stylet receiving chamber and an extended configuration in which the distal end of the stylet is received within the distal opening of the needle lumen sealing the distal opening, wherein, when withdrawn proximally into the stylet receiving lumen, the stylet is coiled within the chamber.

### BRIEF DESCRIPTION OF THE DRAWINGS

FIG. 1 is a partial cross-sectional view of a device according to the present invention.

### DETAILED DESCRIPTION

The present invention, which may be further understood with reference to the following description and the appended drawings, relates to an apparatus and method for obtaining tissue samples and, more particularly relates to EUS-FNA devices. The needle design of the present invention which may be used in substantially all procedures employing EUS-FNA devices further increases the efficacy of EUS-FNA procedures by permitting the procurement of tissue samples from a living body without removing the stylet from the device.

Devices and methods according to the present invention employ an FNA device comprising a stylet which, when retracted proximally thereinto, assumes a storage configuration such as a coiled configuration within a handle of the FNA device eliminating the need to remove the stylet completely from the FNA device when a sample is to be obtained. In this manner, the stylet may be easily retracted to a proximal portion of a needle without having to remove the stylet from the FNA device altogether. It is noted that the use of the term distal herein refers to a direction approaching a target site in a patient in an operative configuration and the term proximal refers to a direction approaching a user of the device (e.g., a physician) with a proximal portion of the device remaining external to the patient.

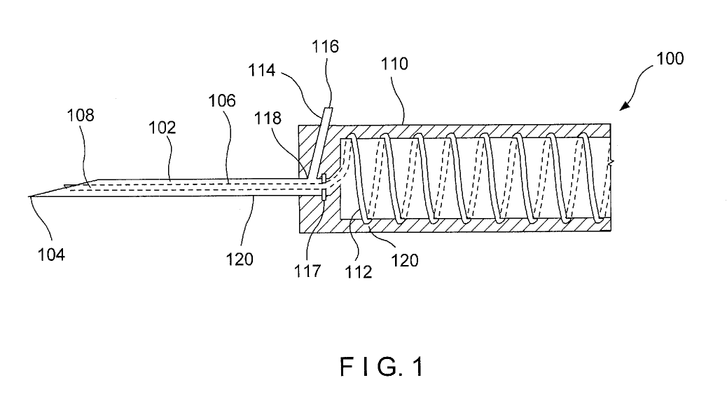

As shown in FIG. 1, a device 100 according to an exemplary embodiment of the present invention for use with an EUS-FNA actuation mechanism (not shown) comprises an elongated hollow body 102 with a puncturing point 104 at a distal end thereof. It is noted however, that the device 100 may take any other shape and may also comprises a blunt distal end without deviating from the scope of the present invention. The elongated body 102 may be composed of any of a variety of suitable materials known in the art such as, for example, stainless steel and nitinol. The elongated body 102 includes a first lumen 106 extending therethrough to a distal opening 108 at the puncturing point 104. A handle 110 formed at a proximal end of the body 102 and having an outer diameter greater than an outer diameter of the elongated body 102 remains outside the body during use. The handle 110 may comprise a second lumen 112 extending therethrough and open to the first lumen 106, a diameter of the second lumen 112 being substantially equivalent to or greater than a diameter of the first lumen 106. In one embodiment, the second lumen 112 follows a substantially coiled path through the handle 110 and may be formed with any number of turns suitable to accommodate a required length of the stylet 120 therethrough. The handle 110 may be integrally formed with the elongated body 102 or, alternatively, may be bonded thereto using any known bonding technique including gluing, insert molding and other permanent or temporary fixation techniques. In the embodiment shown, the handle 110 is connected to the elongated body 102 via a tapered portion 118. It is noted however, that the part of the elongated body 102 which remains outside the body during use may take any shape as would be understood by those skilled in the art. The device 100 also comprises a stylet 120 configured to seal the first lumen 106 extending through during an initial penetration into target body tissue, the stylet 120 minimizing the entry of blood, tissue, etc. into the first lumen 106 before a target sampling site has been reached, as those skilled in the art will understand. An exemplary stylet 120 according to the present invention can be formed of a material known in the art including, but not limited to Nitinol, stainless steel, elgiloy, titanium, tantalum and polymers. The stylet 120 may have shape-memory properties as would be understood by those skilled in the art, the stylet 120 being formed to assume a substantially coiled memorized shape. In such an embodiment, the second lumen 112 may be formed with a substantially cylindrical shape instead of the substantially coiled shape of FIG. 1, an outer diameter of the coiled shape of the stylet 120 being dimensioned to frictionally engage an inner wall of the second lumen 112. It is further noted that the handle 110 may also be formed without a second lumen 112. Rather, the handle 110 may be formed as a hollow substantially cylindrical element having a substantially cylindrical hollow core. In this embodiment, the stylet 120 may be retracted into the handle 110 within which it will assume its shape-memorized coiled configuration, as those skilled in the art will understand. In yet another embodiment of the present invention, the handle 110 may be formed with a grooved patterns molded on an inner wall thereof, the pattern configured to guide the stylet 120 into a retracted (i.e., coiled) configuration. An inner wall of the first lumen 106 and the handle 110 be provided with a hydrophilic coating or other biocompatible lubricious coating to ease insertion of the stylet 120 therethrough.

An outer diameter of a distal portion of the first lumen 106 is only slightly larger than an outer diameter of the stylet 120 so that the stylet 120 substantially seals the first lumen 106 when it is advanced to the distal end of the first lumen 106. In an alternate embodiment, a distal tip of the stylet is of a higher durometer than a proximal portion thereof in order to maintain an overall flexibility of the stylet 120 while still occluding the distal end of the first lumen 106. In one embodiment, the increased durometer portion of the stylet is greater than approximately 2.54 cm. in length, although this value may be changed to affect the flexibility of the stylet accordingly. In an exemplary embodiment of the invention, the inner diameter of the first lumen 106 is substantially constant. In another embodiment, however, the inner diameter of the first lumen 106 may increase at a point proximal of the distal end of the first lumen 106 to house sampled body tissue and facilitate aspiration. Such a device is disclosed in U.S. Provisional Application No. 61/235,465 entitled “Flared Needle for EUS Fine Needle Aspiration Device” filed Aug. 20, 2009, the entire contents of which are incorporated herein by reference. The second lumen 112 is substantially cylindrical and forms a central opening through which the stylet 120 may be retracted. A proximal portion of the first lumen 106 housed within the handle 110 comprises a Y-adaptor leading to a third lumen 114. The third lumen extends out of the handle 110 and comprises an opening 116 at a proximal end thereof for the connection with a syringe (not shown) or other source of aspiration. The opening 116 may be formed with a seal (not shown) to prevent the entry of foreign matter thereinto when not connected to a source of aspiration. The first lumen 106 may terminate within the handle 110 at a location proximal of an opening 118 of the third lumen 114, a proximal end of the first lumen 106 further comprising a valve 117 such as a touhy-borst valve. The touhy-borst valve 117 may be configured to maintain a position of the stylet 120 within the first lumen 106 while permitting advancement and retraction thereof. Alternatively, any other suitable valve may be used in place of the touhy-borst valve 117 including, but not limited to duckbill valves, gel-filled valves, foam-filled valves, ball valves and stopcocks. Accordingly, although portions of the stylet 120 received within the first lumen 106 are constrained to a substantially linear arrangement, portions of the stylet 120 which are drawn proximally into the second lumen 112 assume the coiled shape defined by the coils of the second lumen 112. In one embodiment, a pitch of the coils of the second lumen 112 may preferably be selected to be substantially equal to a diameter of the stylet 120 so that adjacent turns of the coil contact one another and a length of the stylet 120 which may be received within the second lumen 112 is maximized. Furthermore, a length of the second lumen 112 is preferably chosen so that, when the stylet 120 is fully retracted proximally, the distal end of the stylet 120 is withdrawn into the second lumen 112 proximal of the third lumen 114. This moves the distal end of the stylet 120 out of the first lumen 106 permitting the passage of fluids and/or tissue through the third lumen. A proximal end of the third lumen 114 may be connected to a source of negative pressure such as a vacuum pump, syringe, etc. to aspirate sampled tissue through the first lumen into the third lumen 114. A seal (not shown) may also be provided at this proximal end to ensure proper transfer of a vacuum pressure therethrough.

The stylet 120 may be advanced and retracted within the device 100 by actuating an actuating mechanism (not shown) located on a proximal end of the handle 110. The actuating mechanism (not shown) may be formed of a design including, but not limited to, a rotatable portion (not shown) on the handle 110, rotation of the rotatable portion causing distal advancement or proximal retraction of the stylet 120 or a motorized component configured to control movement of the stylet 120. In one embodiment, the actuating mechanism may be a ratchet mechanism or pulley to permit linear actuation of the stylet 120, as those skilled in the art will understand. In an operative configuration, the actuating mechanism is configured to advance the stylet 120 out of the second lumen 112 into the first lumen 106 and through the first lumen 106 until a distal end of the stylet 120 is received in the distal opening of the first lumen 106. When a target tissue site has been reached, the stylet 120 is retracted so that a distal end thereof is located proximal to the opening 118 of the third lumen 114 so that a source of negative pressure applied to the third lumen 114 permits sampling of tissue or other biological matter thereinto. It is noted that although the present invention has been described with respect to a coiled path of the second lumen 112 through the handle 110, the second lumen 112 may follow any path through the handle without deviating from the scope of the present invention.

It is noted that, although the present invention has been described with reference to specific exemplary embodiments, those skilled in the art will understand that various modifications and changes may be made to the embodiments. For example, the exemplary embodiments of the present invention call for a path of the stylet through the handle that is longer than a longitudinal length of the handle itself to permit retraction of the stylet thereinto without removing the stylet from the body. Thus, the stylet may follow any path through the handle without deviating from the scope of the present invention. The specifications are, therefore, to be regarded in an illustrative rather than a restrictive sense.

What is claimed is:

1. A medical instrument, comprising:
a body having a proximal end and a distal end and defining a lumen extending axially therethrough from the proximal end to the distal end;
a stylet removably received within the lumen; and
a handle coupled to the proximal end of the body, the handle comprising an open chamber therewithin sixed and shaped to receive the stylet in a coiled configuration so that an axial length of the chamber is less than a length of the stylet when the stylet is received within the body.

2. The medical instrument according to claim 1, wherein the stylet is biased toward a coiled configuration so that portions of the stylet entering the chamber assume the coiled configuration.

3. The medical instrument according to claim 2, further comprising:
a chamber lumen extending through the handle, the chamber lumen following a substantially coiled path.

4. The medical instrument according to claim 2, wherein the stylet is formed of a shape-memory material with the coiled configuration being a memorized shape of the stylet.

5. The medical instrument according to claim 1, further comprising:
a Y-adaptor at a proximal end of the lumen, the Y-adaptor including a first opening connected to an aspiration lumen and a second opening connected to the chamber.

6. A medical device, comprising:
a needle configured for insertion into a body and defining a needle lumen extending therethrough from a proximal end opening to a distal end opening;
a handle connected to a proximal end of the needle and including a stylet receiving chamber therein, the stylet receiving chamber being open to a proximal end of the needle lumen; and
a stylet movably received within the device between a retracted configuration in which a distal end of the stylet is received within the stylet receiving chamber and an extended configuration in which the distal end of the stylet is received within the distal opening of the needle lumen sealing the distal opening,
wherein, when withdrawn proximally into the stylet receiving lumen, the stylet is coiled within the chamber.

7. The medical device of claim 6, wherein a longitudinal length of the needle lumen is greater than a length of the handle.

8. The medical device of claim 6, further comprising:
a Y-adaptor at a proximal end of the needle lumen including a first branch coupled to the stylet receiving chamber and an aspiration lumen, the aspiration lumen extending to a proximal opening selectively coupleable to a source of aspiration pressure.

9. The medical device of claim 6, wherein the aspiration lumen includes a seal to prevent the entry of foreign matter thereinto when not connected to a source of aspiration pressure.

10. The medical device of claim 6, wherein a distal end of the stylet receiving lumen includes a valve.

11. The medical device of claim 6, wherein the stylet is formed of a shape memory material and wherein a memorized shape of the stylet corresponds to a shape of the coil.

12. The medical device of claim 6, wherein the stylet is formed of Nitinol.

13. The medical device of claim 6, wherein the handle includes a stylet actuating mechanism coupled to a proximal end of the stylet.

14. A method for gathering tissue samples in a body, comprising:
providing a tissue sampling needle having a lumen extending from a proximal end of the needle to a distal end of the needle;
positioning a stylet in an extended configuration in which a distal end of the stylet is received in and occludes a distal opening of the needle lumen;
advancing the needle and stylus simultaneously to a target position within a body;
withdrawing the stylet proximally to a retracted configuration in which at least a portion of the stylet is withdrawn proximally from the needle lumen into a stylet receiving lumen formed in a handle of the device until a distal end of the stylet is located proximally of an aspiration lumen formed in the handle of the device and open to the needle lumen; and
drawing target tissue into the aspiration lumen.

15. The method of claim 14, further comprising:
applying a source of negative pressure to the aspiration lumen to draw tissue thereinto.

16. The method of claim 15, wherein the negative pressure is prevented from being applied to the stylet lumen by a touhy-borst valve located at a distal end of the stylet receiving lumen.

17. The method of claim 14, further comprising:
applying one of a temperature change and electrical energy to the stylet to move the stylet to a coiled configuration.

18. The method of claim 14, wherein the stylet is advanced and retracted by actuating an actuating mechanism of the handle.
